(19) United States
(12) Patent Application Publication
(10) Pub. No.: US 2006/0200186 A1
(43) Pub. Date: Sep. 7, 2006

(54) Adjustable access device for surgical procedures

(76) Inventors: Connie P. Marchek, Foxboro, MA (US); William J. Frasier, New Bedford, MA (US); Anthony R. Carlone, Bristol, RI (US); Katherine Herard, Harrisville, RI (US); Nicholas Pavento, Walpole, MA (US); Timothy A. Beardsley, Kingston, MA (US); John Daniel Malone, Franklin, MA (US); Sara Dziedzic, Braintree, MA (US); Sheryl Frank, Taunton, MA (US); Stephen Connolly, Sharon, MA (US); Sean Selover, Westport, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/325,275

(22) Filed: Jan. 4, 2006

Related U.S. Application Data
(63) Continuation-in-part of application No. 11/073,419, filed on Mar. 4, 2005.

(51) Int. Cl. A61M 29/00

(52) U.S. Cl. 606/191

(57) ABSTRACT

An access device for providing access from a skin incision to a surgical site is described. The access device has a first section having a proximal end and a distal end defining a first path therethrough. The access device has a second section having a proximal end and a distal end defining a second path therethrough, the second section is movable relative to the first section, and the first and second sections cooperate to form a continuous path such that movement of the second section changes the length of the path.

### REFERENCE TO RELATED APPLICATIONS

This application is a continuation-in-part of co-pending U.S. patent application Ser. No. 11/073,419, filed Mar. 4, 2005, entitled Adjustable Access Device for Surgical Procedures” (Marchek et al.) (DEP5486USNP), the specification of which is incorporated by reference in its entirety.

### FIELD OF THE INVENTION

The present invention relates to devices used in surgery. More particularly, the present invention relates to adjustable access devices and a method for providing access to surgical sites.

### BACKGROUND OF THE INVENTION

In minimally invasive surgical procedures, providing access to a variety of working spaces is critical for a successful surgical outcome. For example, in spinal surgery, access devices, comprising generally tubular, open-ended structures, are used to provide access to a surgical site. Depending on the location of the surgical site the access devices may require different configurations and lengths to facilitate the surgical procedure.

In the current state of the art, fixed length tubes are used as access devices. Multiple devices in a variety of lengths are stocked in a kit so that the surgeon may find one of a suitable length for the particular surgery being conducted. However, there are times when a surgeon cannot find the preferred length because the devices are in discrete fixed lengths.

### SUMMARY OF THE INVENTION

The present invention provides an adjustable length access device. The access device defines a path to a surgical site and is adjustable in length to reach the surgical site. The access device may be adjustable in many ways. Incremental or infinite adjustability may be provided in the device. Interlocking sections may be assembled to create an access device of the length desired depending on the patient and location of the surgical procedure. A telescoping device may be assembled from two sliding sections to provide access to the surgical site.

According to a first aspect of the invention, an access device for providing access from a skin incision to a surgical site is provided. The access device has a first section having a proximal end and a distal end defining a first path therethrough. The access device has a second section having a proximal end and a distal end defining a second path therethrough, the second section is movable relative to the first section, and the first and second sections cooperate to form a continuous path such that movement of the second section changes the length of the path.

Another embodiment of the invention is an access device with a hollow tubular body having a proximal end, a middle portion and a distal end, defining a path along a longitudinal axis from the proximal to the distal end, the middle portion reversibly collapsible and expandable along the longitudinal axis.

According to another aspect of the invention, an access device-comprises a first tubular section having a proximal end and a distal end defining a first path therethrough; and a second tubular section having a proximal end and a distal end defining a second path therethrough, the first and second sections cooperating to form a continuous path from the proximal end of the first section to the distal end of the second section, the first section removably connected to the second section.

Another embodiment of the invention is an adjustable access port comprising a first section having a fixed cross section and a proximal end and a distal end defining a first path therethrough, the first path having a length and a width, and an extension member operably coupled with the first section and adjustable relative to the first section to change the length of the first path, while the width of the first path remains fixed.

According to still another aspect of the invention, a method of accessing a surgical site in a patient is provided. The method involves determining the distance from the skin incision to proximate a vertebrae, adjusting the length of the tubular access device to the length determined, positioning the access device through the skin incision to proximate the vertebrae.

### BRIEF DESCRIPTION OF THE FIGURES

These and other features and advantages of the present invention will be more fully understood by reference to the following detailed description in conjunction with the attached drawings in which like reference numerals refer to like elements through the different views. The drawings illustrate principals of the invention and, although not to scale, show relative dimensions.

FIG. 1A illustrates an adjustable access system including modular connecting sections according to an embodiment of the invention;

FIG. 1B illustrates an exploded view of the embodiment of the invention shown in FIG. 1A;

FIGS. 1C-1E illustrate the adjustable access system of FIG. 1A including various configurations of the distal tip;

FIG. 2A illustrates an adjustable access system including telescoping sections according to another embodiment of the invention;

FIG. 2B illustrates the telescoping sections of the access device in FIG. 2A in an elongated position;

FIG. 2C illustrates an endview of the adjustable access system of FIG. 2A;

FIG. 2D illustrates an alternate embodiment of the adjustable access system of FIG. 2A in the elongated position;

FIG. 2E illustrates the alternate embodiment of FIG. 2D in the closed position;

FIG. 2F illustrates an endview of the alternate embodiment of FIG. 2D;

FIG. 2G illustrates an exploded view of an alternate embodiment of an adjustable access system.

FIG. 2H illustrates a side view of the assembled embodiment of FIG. 2G.

FIG. 2I illustrates a cross-section view of the assembled embodiment of FIG. 2H.

FIG. 3A illustrates another embodiment of an adjustable access device in an elongated position including two telescoping sections with discrete locking intervals;

FIG. 3B illustrates the adjustable access device of FIG. 3A in a partially elongated position;

FIG. 4 illustrates another embodiment of an adjustable access device including multiple independent extension members;

FIG. 4A illustrates an alternate embodiment of an adjustable access device with extension members in an elongated position.

FIG. 4B illustrates a cross-section taken along the longitudinal axis of the embodiment shown in FIG. 4A.

FIG. 4C illustrates a cross-section taken transverse to the longitudinal axis of the embodiment shown in FIG. 4A.

FIG. 4D illustrates a perspective view of an extension member of the embodiment shown in FIG. 4A.

FIG. 4E illustrates a top view of the extension member shown in FIG. 4D.

FIG. 5 illustrates another embodiment of an adjustable access device including two threaded sections.

FIG. 6A illustrates another embodiment of an adjustable access device including a reversibly collapsible and expandable section in a fully expanded position; and

FIG. 6B illustrates the adjustable device in FIG. 6A in a fully collapsed position.

### DETAILED DESCRIPTION OF THE INVENTION

The present invention provides an adjustable access device for accessing a surgical site during surgery. The present invention will be described below relative to an illustrative embodiment. Those skilled in the art will appreciate that the present invention may be implemented in a number of different applications and embodiments and is not specifically limited in its application to the particular embodiments depicted herein.

The adjustable access device of an illustrative embodiment of the invention may be used in spinal surgery, for example, during a discectomy or microdiscectomy procedure to remove damaged disc material from the spine, though one skilled in the art will recognize that the invention can be used with any surgical instrument in any surgical procedure that requires minimally invasive access. Examples of surgical procedures suitable for employing the access device of the present invention include, but are not limited to, insertion of interbody fusion devices, bone anchors, fixation devices, including rods, plates and cables, artificial disks and hip stems. The access device may include a port or other device such as a retractor having a fixed cross-section, ie. not expandable, while allowing for adjustability in the length of the device. The access device can be used to position any suitable implant, instrument and/or other device in any suitable procedure where guidance of the implant, instrument and/or device is used.

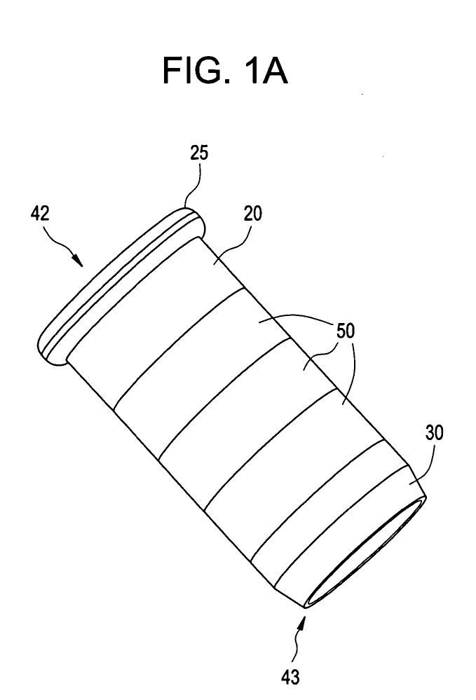

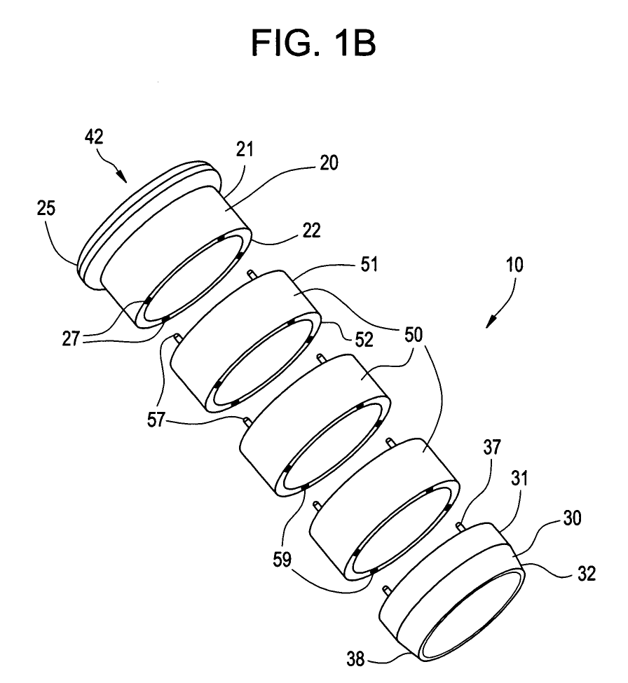

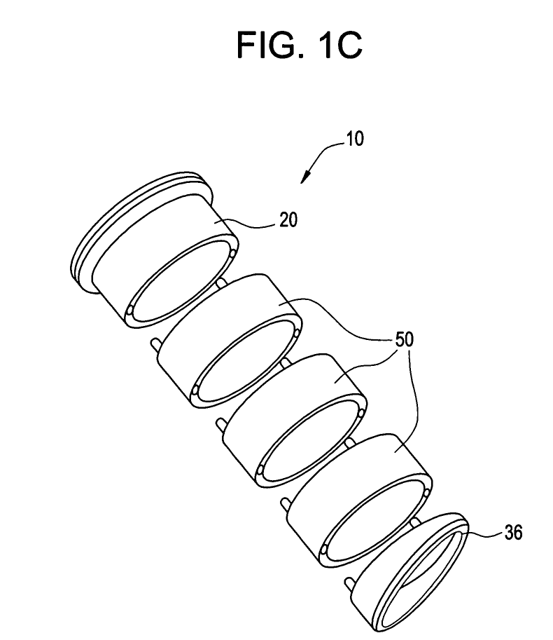

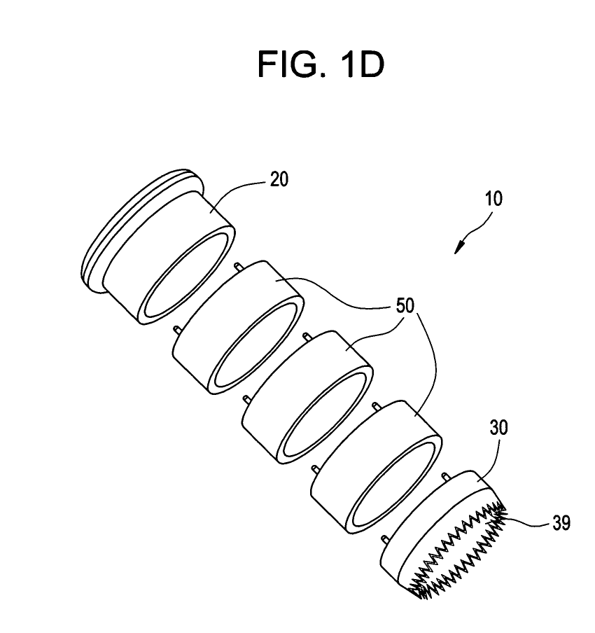

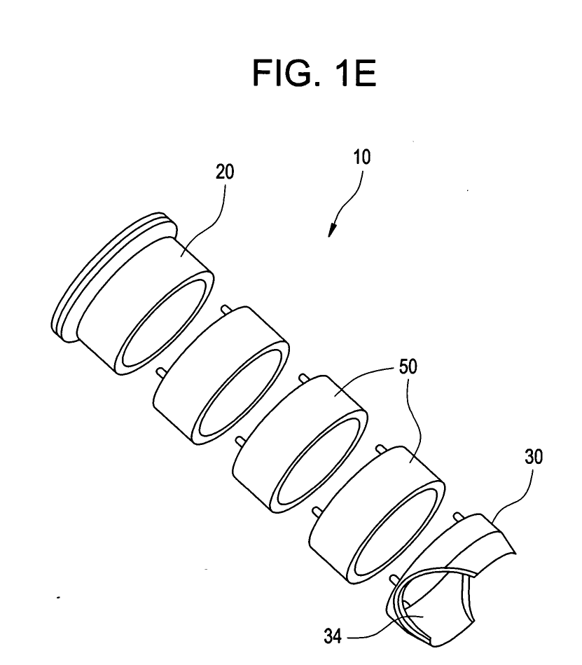

Referring to FIG. 1, an access device 10 of an illustrative embodiment of the invention is shown that provides adjustability in its length to access a surgical site during performance of a surgical procedure. The illustrative access device 10 has a generally hollow tubular body suitable for insertion in a patient's body. The access device 10 has a first section 20 and a second section 30, cooperating to define a continuous path 42 extending from a proximal end 41 of the access device to a distal end 43 of the access device. The path 42 provides access to a surgical site adjacent to or in the vicinity of the distal end 43 of the device. The first and second sections are removably connected. The first section 20 of the device has a hollow tubular body having a proximal end portion 21 and distal end portion 22. The proximal end portion may have an outer lip 25 with a connection for attaching to a mounted arm. The distal end portion has connecting features 27 to connect with another hollow tubular section. The first section 20 defines a proximal portion of the path 42a. The second section 30 of the device has a hollow tubular body having a proximal end portion 31 and a distal end portion 32. The proximal end portion 31 has complementary connecting features 37 to the connecting features 27 on the distal end of the first section. Examples of connecting features include pins and holes, threads, slots or tabs. One skilled in the art could use any known connecting feature to removably connect the two sections together. The distal end portion 32 may have a chamfer 38 to aid in insertion of the device. The distal end may have alternate configurations to match certain anatomic structures depending on the location of the surgical site. For example a U-shaped cutout may be located on one side of the distal end of the second section to fit over anatomic structures and provide a secure pathway through the access device to the surgical site.

An intermediate section 50 may be assembled between the first section 20 and the second section 30 of the device. Intermediate section 50 has a hollow tubular body having a proximal end portion 51 and a distal end portion 52 defining a path between the end portions. The proximal end portion 51 has connecting features 57 to complement the connecting features 27 on the distal end of the first section. The distal end portion of the intermediate section has connecting features 59 complementary to the connecting features 37 on the proximal end portion of the second section or another intermediate section. The connecting features may have a locking mechanism to lock each section together to allow the assembled device to be removed from the surgical site in one piece. Examples of locking mechanisms include but are not limited to, threaded pins with corresponding mating threaded holes or hooks with corresponding J-slots. Another example of a locking mechanism includes a separate pin that each section slides over and a nut or other element secured to the pin after the last section is assembled locking all the sections together. Other examples of locking mechanisms may be used that are known to one skilled in the art.

The diameter of the distal end portion of the first section, the entire intermediate section and the proximal end portion of the second section may be the same defining a path having a constant diameter. In an alternate embodiment the path may be tapered. The illustrative access device may be used for retaining soft tissue away from a surgical site and/or guiding a surgical instrument, device and/or implant, though one skilled in the art will recognize that the access device may comprise any suitable device defining a path or channel.

As shown, the access device 10 is formed by a tubular body, though one skilled in the art will recognize that the tubular body can have any size, shape, configuration and number of side walls. The access device can be any suitable device defining a path for providing access to a surgical site. The access device can have any suitable cross-section, for example, circular, oval or rectilinear and is not limited to the cylindrical cross-section shown in the illustrative embodiments. An exemplary adjustable access device would span lengths of approximately 35 mm to 120 mm.

The intermediate sections 50 may be provided in varying lengths such that multiple intermediate sections can be assembled to the first and second sections to form an access device of the length needed by the surgeon to reach the surgical site. The first section 20 may come in one standard size (length and width) with an outer lip having a connection for attaching to an arm to hold the access device in place during the surgical procedure. The second section 30 may come in one standard size (length and width) as well but there may be multiple embodiments having varying configurations of the distal end portion 32. The alternate configurations may vary depending on interfacing with bone and the various anatomical structures that may be present at the surgical site to facilitate positioning of the access device to create a path to the surgical site. For example, as shown in FIG. 1C, the distal end 32 of section 30′ may be angled 36 to match the curvature of surface of the posterior lamina. Alternately, the section 30″ may have a distal end shaped to engage a part of the surgical site to prevent slippage, and can optionally, as shown in FIG. 1D, include teeth 39 or other suitable feature formed on an outer surface for engaging a part of the surgical site. In another configuration shown in FIG. 1E the section 30′″ may have a distal end having a curvature 34 to match the curvature of the anterior surface of a vertebral body.

The tubular body of the access device can be rigid, semi-rigid or flexible, and can have any suitable size, shape and configuration suitable for defining a working channel and/or access to a surgical site. In the illustrative embodiment, the tubular body is straight to define a straight channel therethrough. Alternatively, the intermediate section of the tubular body can be curved or have any other suitable shape to define a curved or otherwise shaped trajectory. The tubular body is not limited to a tubular structure having closed sidewalls and can be any component that defines a path, including an open channel.

The access device can be formed of any suitable surgical material, such as, but not limited to, plastic and surgical stainless steel.

According to the illustrative embodiment, an adjustable access device kit may be composed of a plurality of modular sections 20, 30, 50 that can be connected to form an access device 10, of the length needed by the surgeon to access the surgical site. FIG. 1B shows an exploded view of the different modular sections connected together to create an adjustable length device to provide access to a patient according to one embodiment of the invention. At least two sections are connected to form an adjustable access device. Multiple intermediate sections may be assembled between the first and second sections to create an access device of the length desired by the surgeon. The kit may also include a plurality of sections offered in a variety of widths as well to provide the surgeon with more options for accessing the surgical site.

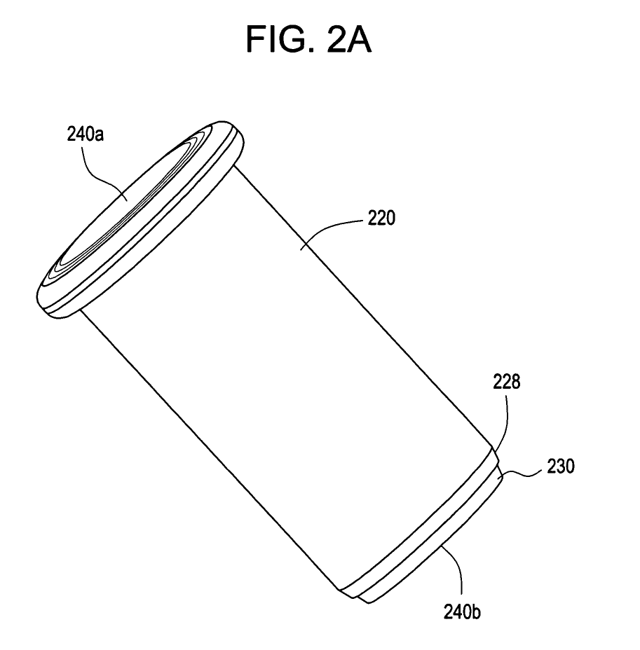

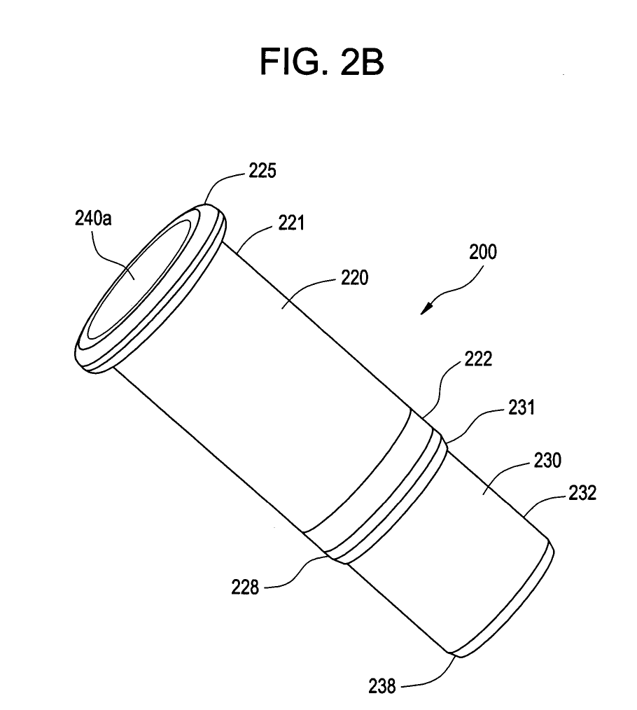

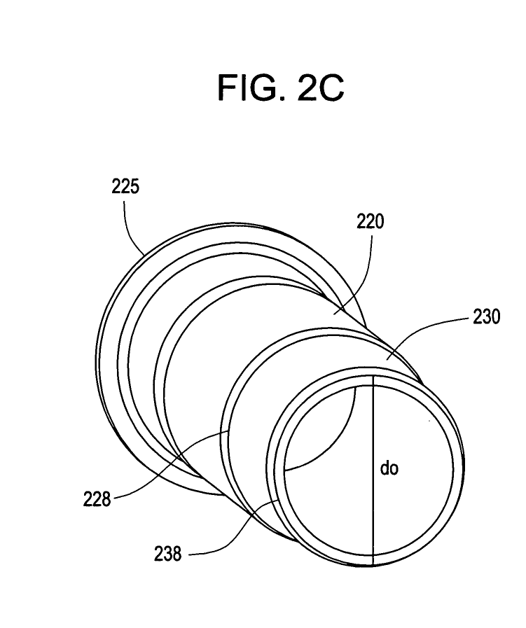

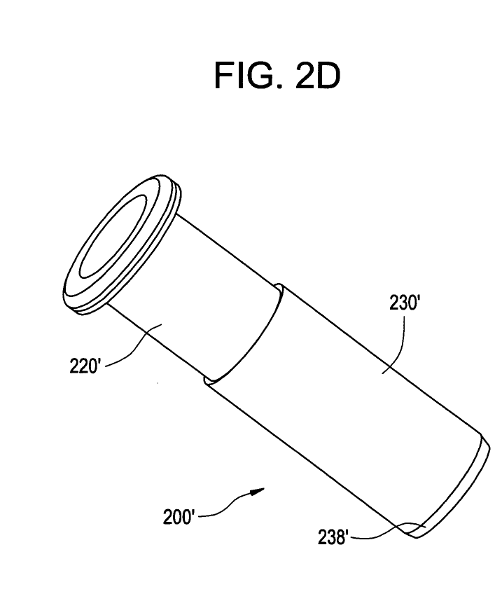

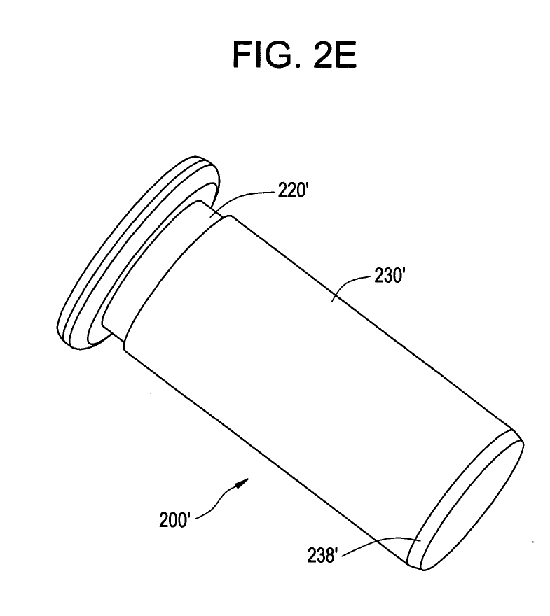

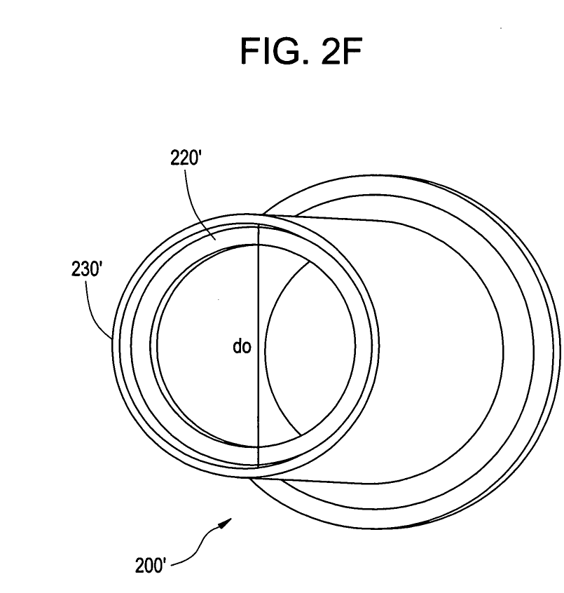

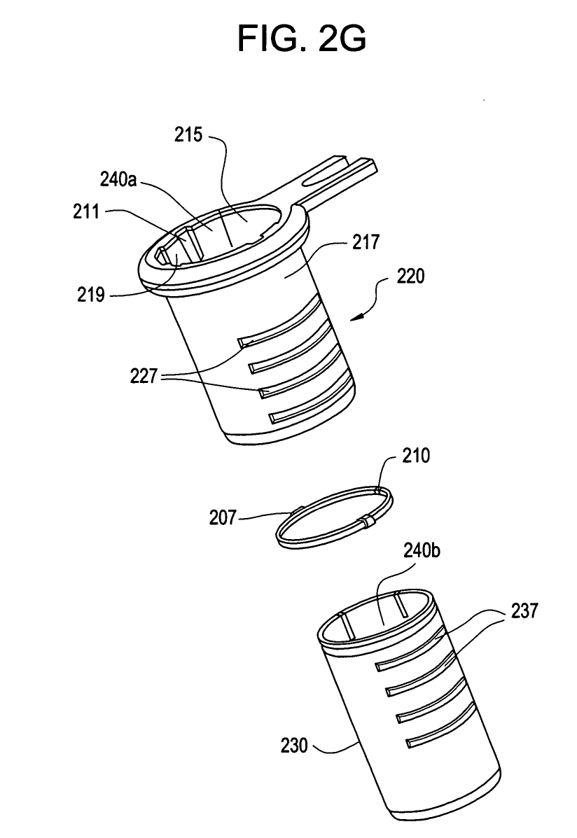

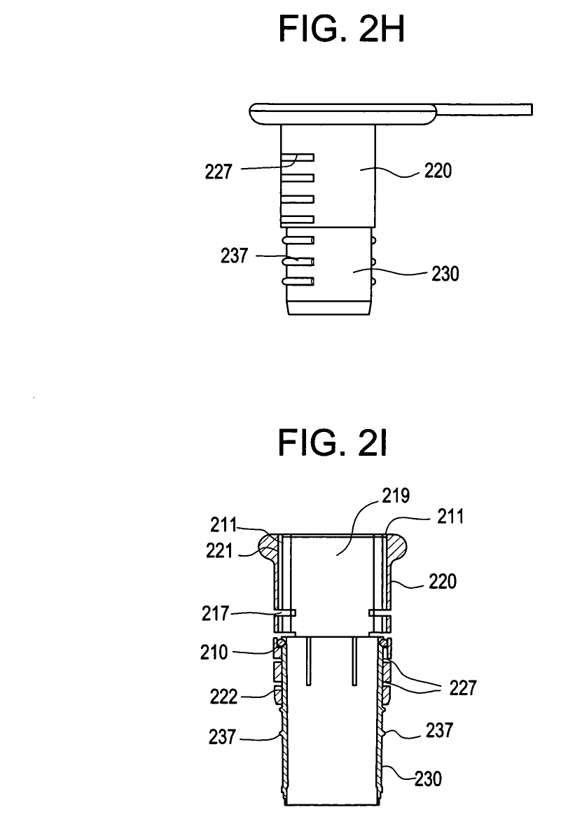

The adjustable access device of the illustrative embodiment of the invention can comprise any suitable means for adjusting the length of an access device. For example, as shown in FIGS. 2A-2B, the access device 200 may comprise a first proximal section 220 and a second distal section 230 positioned along a longitudinal axis. The proximal section 220 forms a hollow channel defining a path 240a from the proximal end 221 to the distal end 222 of the section. The proximal section has an inner diameter dᵢ along the path. The proximal end of the proximal section may have an outer lip 225 having a connection for attachment to a mounted arm. The distal end of the proximal section may have a chamfer 228 for ease of insertion through a skin incision.

The second distal section 230 forms a hollow channel defining a second path 240b from the proximal end 231 to the distal end 232 of the second section. The distal end 232 of the second distal section may have a chamfer 238 for ease of insertion if inserted in the elongated state. The distal end may also be configured to interface with bone and the various anatomical structures that may be present at the surgical site to facilitate positioning of the access device to create a path to the surgical site. For example, the distal end 232 may be shaped to engage a part of the surgical site, such as a vertebral structure, and can optionally include teeth or other suitable feature formed on an outer surface for engaging a part of the surgical site, such as a vertebra.

The second section 230 is movable relative to the first section 220 such that the length of the path changes with movement of the second section. As shown in FIG. 2C, the second distal section 230 has an outer diameter do slightly smaller than the inner diameter dᵢ of the proximal section 220 to allow the distal section to move within the hollow channel of the proximal section. The distal section can be advanced through the proximal section to adjust the access device to the length that the surgeon needs to reach the surgical site. The proximal and distal sections can be locked into the place when adjusted to the desired length by using a luer-lock type connection or a molded-in snap feature.

In an exemplary embodiment illustrated in FIGS. 2G-I, the first section 220 may have an inner surface 215 and outer surface 217. The inner surface 215 defines a first path 240a. The second section 230 may be operably coupled with the first section and adjustable relative to the first section 220 to change the length of the first path 240a, while the width of the first path 240a remains fixed. The first section 220 may have one or more openings 227 extending transverse to the longitudinal axis of the section and spaced apart along the longitudinal axis for coupling with the second section 230. The inner surface 215 of the first section 220 may have a recess 219 extending from the proximal end 221 to the distal end 222 configured to provide for adjustment of the second section 230 relative to the first section 220. The second section 230 may have tabs 237 projecting from the outer surface for engagement with the openings 227 of the first section 220 to fix the position of the second section 230 relative to the first section 220. The tabs 237 of the second section 230 may be aligned with the recess 219 of the first section 220 allowing the second section to move relative to the first section along the longitudinal axis. The second section 230 may be rotated so that tabs 237 may be positioned in or out of the opening 227 when the access device has obtained the desired length to fix the position of the second section 230 relative to the first section 220. The second section 230 may be rotated either by hand or by an instrument (not shown).

A retaining member 210 may be operably positioned around the proximal end of the second section 230 to retain the second section within the first section 220 during adjustment. The retaining member 210 may have posts 207 for engaging a channel 211 within the inner surface 215 of the first section 220. The channel 211 may extend from the proximal end 221 of the first section 220 towards the distal end 222. The channel 211 may be open at the proximal end to receive the post 207 of the retaining member 210 and closed at the distal end to prevent the retaining member 210 from passing through the first section 220. The retaining member 210 allows for rotation of the second section 230 relative to the retaining member 210 and the first section 220.

In an alternate embodiment 200′ shown in FIGS. 2D-E, the device has a distal section 230′ and a proximal section 220′. The distal section 230′may have an inner diameter dᵢ, slightly larger than the outer diameter, dₒ, of the proximal section 220′ allowing the distal section to move along the outer surface of the proximal section. An end view of this embodiment is shown in FIG. 2F. Other types of locking connections known to one skilled in the art can also be used to lock the sections together in the adjusted position.

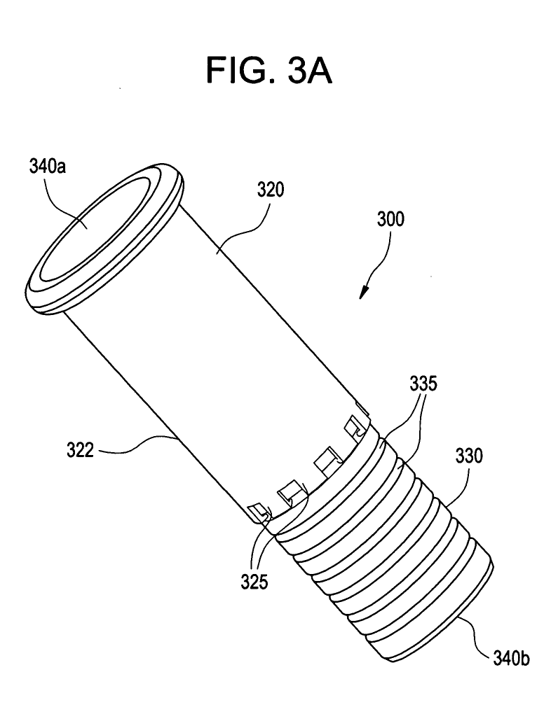

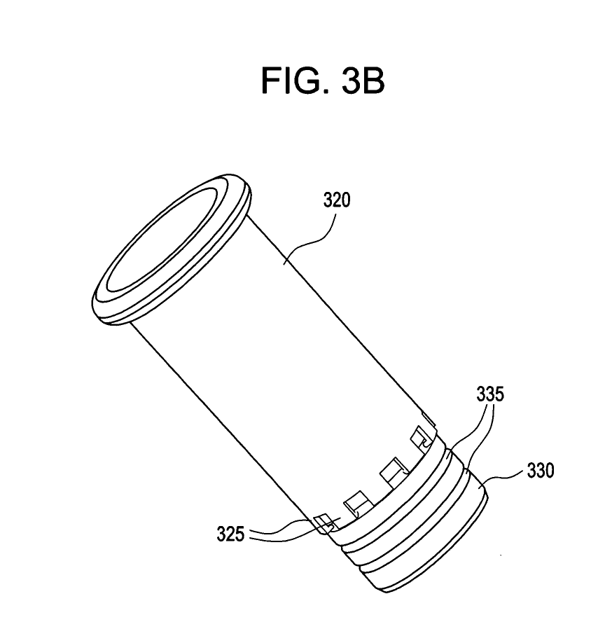

An alternate embodiment of this invention is shown in FIG. 3A and FIG. 3B. The access device 300 has a first proximal section 320 having a hollow tubular body defining a first path 340a and a second distal section 330 having a hollow tubular body defining a second path 340b extending along a longitudinal axis of the device. The proximal section 320 has an inner diameter defining the perimeter of the path. The distal section 330 has an outer diameter slightly smaller than the inner diameter of the proximal section such that the distal section fits within the hollow tubular body of the proximal section. Alternatively, the distal section may have an inner diameter dᵢ, slightly larger than the outer diameter, dₒ, of the proximal section allowing the proximal section to move within the hollow channel of the distal section. The outer surface of the distal section has individual grooves 335 cut circumferentially around the longitudinal axis. The inner surface of the proximal section may have a ball plunger at the distal end to engage the grooves of the distal section. Alternately, the distal end 322 of the proximal section may have spring-loaded fingers 325 to engage the grooves 335 on the distal section to hold the two sections together at a fixed length. One skilled in the art will understand that the surface where the grooves are can be interchanged. For example, the inner surface of the proximal section may have grooves cut circumferentially around the longitudinal axis and the outer surface of the distal section may have a ball plunger or spring loaded fingers to engage the grooves.

The distal end may also be configured to interface with bone and the various anatomical structures that may be present at the surgical site to facilitate positioning of the access device to create a path to the surgical site. For example, the distal end 330 may be shaped to engage a part of the surgical site, such as a vertebral structure, and can optionally include teeth or other suitable feature formed on an outer surface for engaging a part of the surgical site, such as a vertebra.

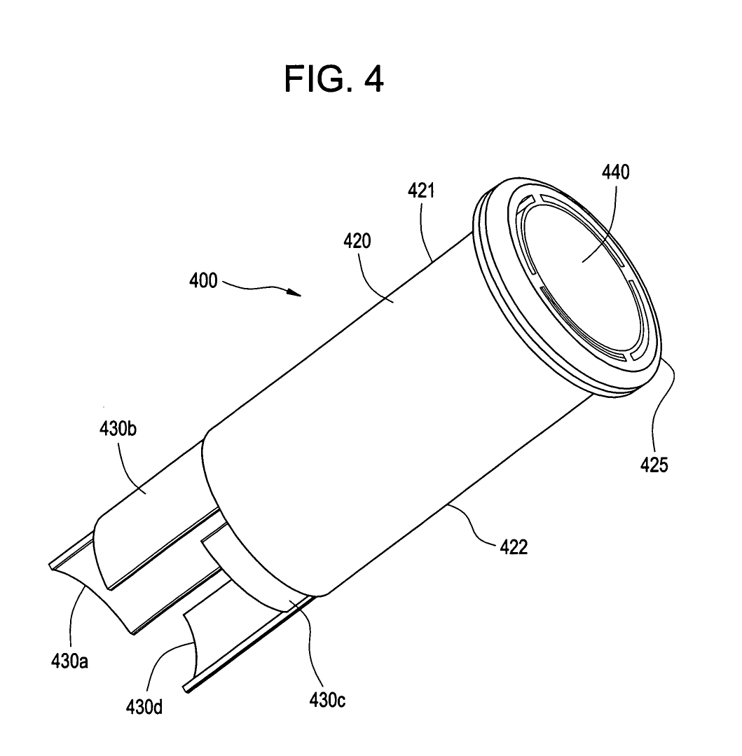

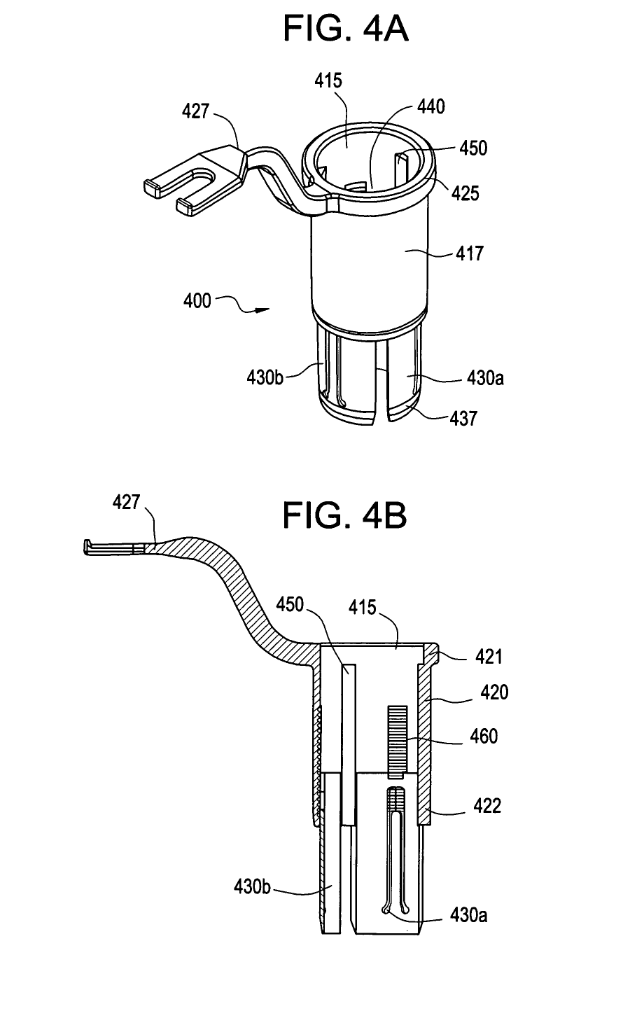

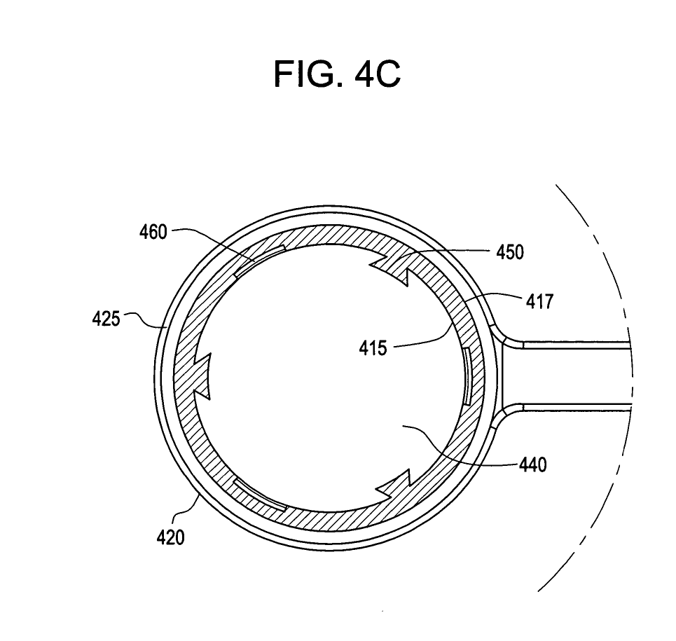

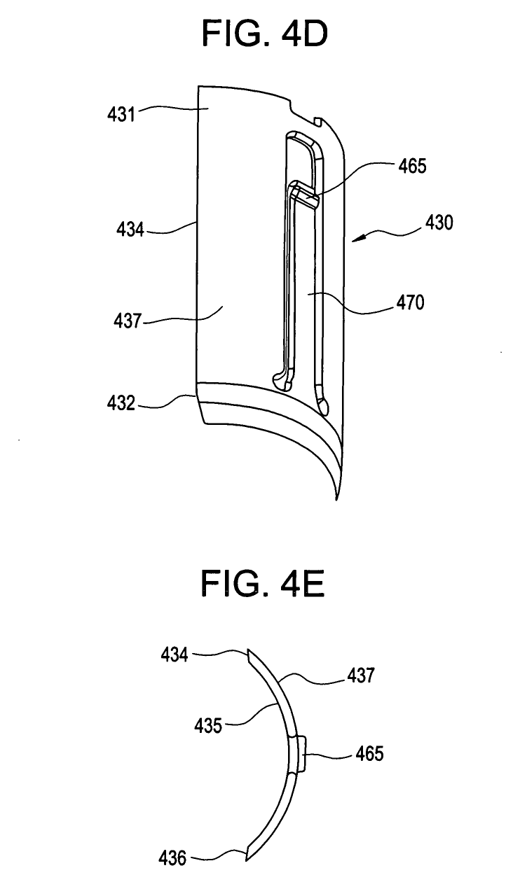

In an alternate embodiment of the invention shown in FIGS. 4-4E, the access device 400 has a first section 420 having a fixed cross-section with a hollow channel extending from the proximal end 421 to the distal end 422 defining a path 440 therethrough. The first path 440 has a length and a width; the width is not adjustable. The first section 420 has an inner surface 415 and an outer surface 417. The section 420 may have an outer ring 425 extending from the proximal end 421. Outer ring 425 may have a connection 427 for a mounted arm attachment. In one embodiment the first section 420 may have a generally tubular shape.

A number of individual extension members 430a, 430b, 430c, 430d, may be operably coupled with the first section 420 and adjustable relative to the first section to change the length of the first path 440, while the width of the first path remains fixed. In one embodiment, the extension members 430 may slide within slots 410 in section 420 to adjust the length of the path 440 defined by the inner surface 415 of section 420. The section 420 may have slots 410 between the inner surface 415 and outer surface 417 extending longitudinally from the proximal end 421 to the distal end 422 for the extension members 430 to slide along. Each extension member 430 may be adjusted individually to the length desired by the surgeon using an instrument or by hand while the access device 400 is in situ.

Alternately as shown in FIG. 4A, the section 420 may have rails 450 projecting from the inner surface 415 of the section 420, extending longitudinally to the distal end 422 and spaced apart for coupling with the extension members 430. The length of the rails 450 may be varied depending on the length of the extension member 430 and need not extend the entire length of the first section 420. In one embodiment the rails 450 may have a dovetail configuration for coupling and guiding the extension members 430 during adjustment relative to the first section 420. One skilled in the art will recognize that other configurations may be used to guide the extension members such as mortise and tenon, pin and groove, or other complementary mating shapes.

The inner surface 415 of the first section 420 may optionally include one or more stops for fixing the extension members 430 at a position along the longitudinal axis of the first section 420. In the exemplary embodiment illustrated in FIG. 4B, for example, the first section 420 includes a plurality of stops in the form of teeth 460 aligned longitudinally along the first section 420. The teeth 460 are configured to engage a projection 465 on the extension member 430 as described in more detail below.

The extension members 430 extend from a proximal end 431 to a distal end 432 along a longitudinal axis. The extension member 430 may have an inner surface 435 and an outer surface 437. The inner surface 435 of the extension member 430 in cooperation with the inner surface 415 of the first section 420 can further define and adjust the length of the path 440. The outer surface 437 of the extension member 430 in cooperation with the outer surface 417 can contact tissue, to inhibit the tissue from interfering with the path 440. The outer surfaces 417, 437 may have surface texturing, for example, grooves, surface roughening, or coatings to aid in retracting the tissue from entering the path 440. The distal end 432 of the extension member 430 may be chamfered for ease of insertion. Alternately the distal end 432 may be contoured to match an anatomical structure at the surgical site.

Referring to FIGS. 4D-E, the extension member 430 may include a flexible tab 470 that facilitates adjustment of the extension member 430 relative to the first section 420 and cooperates with one or more of the stops provided on the first section 420 to fix the extension member relative to the first section 420. The flexible tab 470 moves from a first position in which it is coplanar with the extension member 430 to a second position in which it is transverse to the extension member 430. When in the first position, the extension member 430 may be fixedly coupled relative to the first section 420. When in the second position, the extension member 430 may be disengaged and adjusted relative to the first section 420. The flexible tab 470 may be biased in the first position.

The tab 470 may include a projection 465 that is configured to engage the first section 420 for example one or more of the stops provided on the first section 420 to fix the extension member 430 relative to the first section 420. The projection 465 may be sized and shaped to engage a stop on the first section 420. In the exemplary embodiment, the projection 465 has a triangular shaped cross-section configured to engage the teeth 460 of the first section 420. In the exemplary embodiment, when the tab 470 is in the first position, the projection 465 engages one of the teeth 460 of the first section 420. The projection 465 disengages from the teeth 460 when the tab 470 is in the second position, allowing the extension member 430 to be adjusted relative to the first section 420.

The extension member 430 may be sized and shaped to fit between the rails 450 of the first section 420. In an exemplary embodiment shown in FIG. 4E, the sides 434, 436 of the extension member 430 may be contoured to slidably engage the rails 450 of the first section 420 and guide the extension member 430 during adjustment relative to the first section 420. Each extension may be independently adjusted such that the length of the first path may be different along at least one extension member.

Various locking mechanisms may be used to lock the extension members in place, including ratchet teeth, ball detents, friction fits, cams, set screws, or j-shaped slots. One skilled in the art could use any known locking mechanism to lock the extension members in place. Examples of adjustable extension members and locking mechanisms are described in published US patent application 2005/0137461A1 filed on Mar. 24, 2004 which is hereby incorporated by reference in its entirety.

Alternately each extension member 430a, 430b, 430c, 430d may be connected by a deformable polymer or mesh.

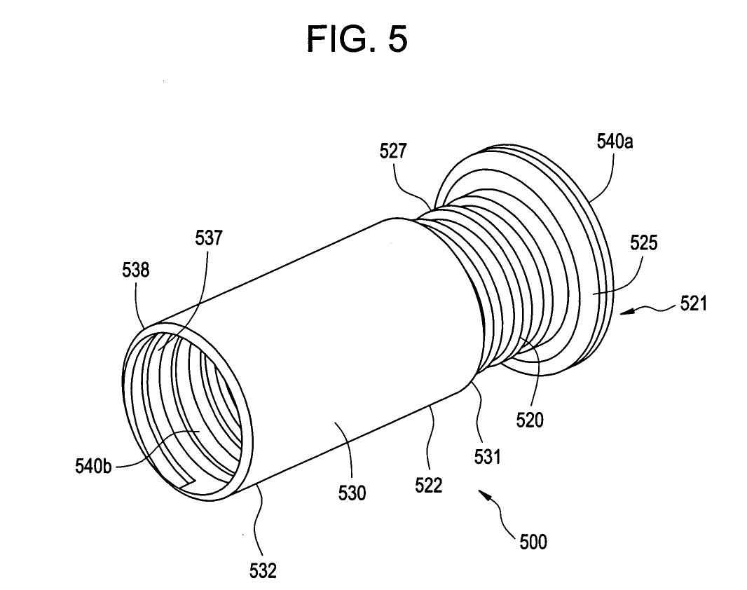

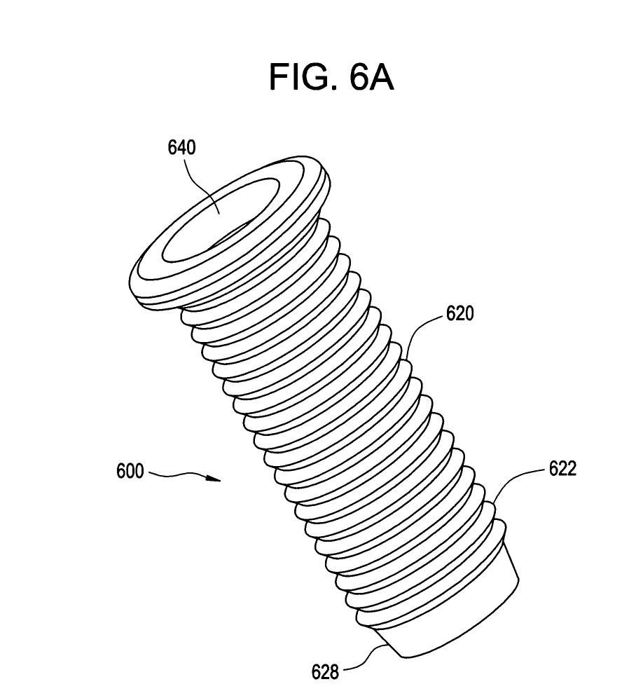

According to another embodiment of the invention, shown in FIG. 5, the access device 500 may have a proximal section 520 having a hollow channel defining a first path 540a from the proximal end 521 to the distal end 522 and a distal section 530 having a hollow channel defining a second path 540b from the proximal end 531 to the distal end 532. The outer surface of the proximal section has threads 527. The inner surface of the distal section has a mating thread 537 to the thread on the outer surface of the proximal section. The distal section moves along the threads to adjust the length of the path created by the access device to the desired length. Alternatively, the outer surface of the distal section may contain threads, and the proximal section would have mating threads on the inner surface. The proximal end of the proximal section may have an outer lip 525 with a connection for attaching to a mounted arm. The distal end of the proximal section and the distal section may have a chamfer 538 for ease of insertion into the surgical incision. The distal end may also be configured to interface with bone and the various anatomical structures that may be present at the surgical site to facilitate positioning of the access device to create a path to the surgical site. For example, the distal end 538 may be shaped to engage a part of the surgical site, such as a vertebral structure, and can optionally include teeth or other suitable feature formed on an outer surface for engaging a part of the surgical site, such as a vertebra.

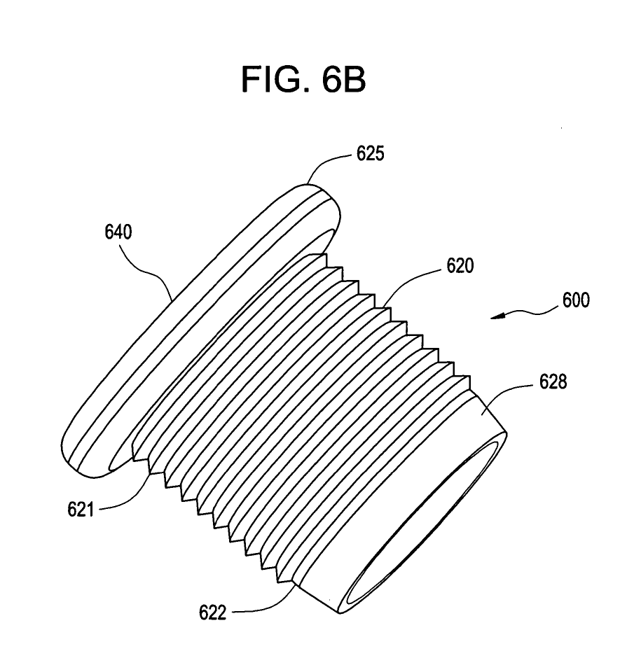

According to another embodiment of the invention, an access device 600 as shown in FIGS. 6A-6B, may have one section 620 having a proximal end 621 and a distal end 622 and a hollow channel defining a path 640 extending therethrough along a longitudinal axis. The access device is made of a material that keeps its shape when loaded in a direction normal to the longitudinal axis of the device but which collapses when loaded in a direction axial to the longitudinal axis of the device. The device is reversibly collapsible in that it can be returned to its original shape. The device in its collapsed state as shown in FIG. 6B has a length L_{c} and when fully extended has a length Le as shown in FIG. 6A. The device is also adjustable to various lengths between the fully extended and collapsed states. The device may have an outer ring 625 at the proximal end with an attachment for a mounting arm. The distal end of the device may have a chamfer 628 for ease of insertion. The distal end may also be configured to interface with bone and the various anatomical structures that may be present at the surgical site to facilitate positioning of the access device to create a path to the surgical site. For example, the distal end 628 may be shaped to engage a part of the surgical site, such as a vertebral structure, and can optionally include teeth or other suitable feature formed on an outer surface for engaging a part of the surgical site, such as a vertebra.

A method of the present invention for accessing a surgical site in a patient can be performed using any number of access devices. The method can also be performed using only some of the method steps disclosed herein, and/or using other methods known in the art. The surgeon determines the depth of an access pathway from a skin incision to proximate the site to perform the surgery. The length of the tubular access device is adjusted to the determined depth and positioned through the incision to define the access pathway from the skin incision to proximate the surgical site. The length of the access device may be locked at the determined length. Alternately, the surgeon may adjust the length of the device once the device is positioned through the incision.

In one embodiment, a method of accessing a surgical site using an adjustable access device includes positioning through a skin incision to a surgical site proximate a vertebrae, an adjustable access device having a first section with a fixed cross-section and an extension member operably coupled with the first section and adjusting the extension member within the incision relative to the first section to change the length of the first path, while the width of the first path remains fixed.

The method may further include adjusting a number of extension members within the incision to adjust the length of the first path. Alternately, the user may adjust at least one extension member to a different length than the other extension members.

The present invention has been described relative to an illustrative embodiment. Since certain changes may be made in the above constructions without departing from the scope of the invention, it is intended that all matter contained in the above description or shown in the accompanying drawings be interpreted as illustrative and not in a limiting sense. For example, one skilled in the art will recognize that the instrument of the illustrative embodiment of the invention is not limited to use with polyaxial screws and can be used with any suitable implant or procedure for any suitable orthopedic system.

It is also to be understood that the following claims are to cover all generic and specific features of the invention described herein, and all statements of the scope of the invention which, as a matter of language, might be said to fall therebetween.

1. An access device for providing access to a patient during surgery, comprising
a first section having a proximal end and a distal end defining a first path therethrough; and
a second section having a proximal end and a distal end defining a second path therethrough, the second section is movable relative to the first section, the first and second sections cooperating to form a continuous path such that movement of the second section changes the length of the path.

2. The access device of claim 1, wherein the second section moves relative to the first section in a direction substantially parallel to a longitudinal axis of the first body.

3. The access device of claim 1, wherein the first and second sections are substantially tubular.

4. The access device of claim 1 further comprising a locking mechanism to lock the position of the second section with respect to the first section.

5. The access device of claim 1, wherein the first section has an inner diameter and the second section has an outer diameter that is smaller than the inner diameter of the first section wherein the second section fits within the first section.

6. The access device of claim 1, further comprising an outer lip extending from the proximal end of the first section having an attachment mechanism for connecting to an arm.

7. The access device of claim 1, wherein the second section has a chamfer around the distal end for ease of insertion.

8. The access device of claim 7, wherein the second section has an outer surface that is smooth.

9. The access device of claim 7, wherein the second section has an outer surface that has circumferential grooves extending along the longitudinal axis.

10. The access device of claim 9, wherein the first section has an inner surface having a ball plunger for engaging the grooves along the outer surface of the second section to lock the two sections in a selected position.

11. The access device of claim 9, wherein the first section has spring-loaded fingers extending from the distal end for engaging the grooves along the outer surface of the second section to lock the two sections in a selected position.

12. The access device of claim 1, wherein the first section has an outer diameter and the second section has an inner diameter that is larger than the outer diameter of the first section wherein the second section fits over the first section.

13. The access device of claim 12, wherein the first section has a longitudinal axis and an outer surface that is threaded along the longitudinal axis.

14. The access device of claim 13, wherein the second section has a longitudinal axis and an inner surface having mating threads along the longitudinal axis that cooperate with the threads on the outer surface of the first section to move the second section along the longitudinal axis of the first section.

15. The access device of claim 5, wherein the first section has a protrusion from the inner surface and the second section has a channel extending from the proximal end for the protrusion to slide within.

16. An access device, comprising a hollow body having a proximal end, a middle portion and a distal end, defining a path along a longitudinal axis from the proximal to the distal end, the middle portion reversibly collapsible and expandable along the longitudinal axis.

17. The access device of claim 16, wherein the middle portion is collapsible under a load applied parallel to the longitudinal axis of the device.

18. The access device of claim 16, wherein the proximal end portion has an outer lip with attachment mechanism for connecting to an arm.

19. An access device for providing access to a patient during surgery, comprising
a first tubular section having a proximal end and a distal end defining a first path therethrough; and
a second tubular section having a proximal end and a distal end defining a second path therethrough, the first and second sections cooperating to form a continuous path from the proximal end of the first section to the distal end of the second section, the first section removably connected to the second section.

20. The access device of claim 19, wherein the distal end of the first section and the proximal end of the second section have interlocking features to connect the sections.

21. The access device of claim 20, wherein the interlocking features are selected from the group consisting of pins and holes, threaded pins and threaded holes, tabs and slots, tongue and groove, and hooks and j-slots.

22. The access device of claim 20, wherein the interlocking features are adapted to allow the device to be withdrawn from the patient in one piece.

23. The access device of claim 19, further comprising a third tubular section having a proximal end and a distal end defining a third path therethrough, the third section removably connected to the second section to form a continuous path from the proximal end of the first section to the distal end of the third section.

24. A kit for accessing a surgical site in a patient comprising:
a plurality of modular hollow sections connectable to form an access device defining a path of a desired length.

25. A method of accessing a surgical site in a patient comprising the steps of:
determining the depth of an access path from a skin incision to proximate a vertebrae;
adjusting the length of a tubular access device to the determined depth; and
positioning the access device through the incision to define the access path from the skin incision to proximate the vertebrae.

26. The method of claim 25, wherein adjusting the length of the tubular access device further includes moving a second section of the access device relative to a first section of the access device.

27. The method of claim 25 further comprising, locking the length of the access device at the determined depth.

28. The method of claim 25, wherein adjusting the length of the tubular access device occurs after the device is positioned through the incision.

29. The access device of claim 19 further comprising a retaining member adapted to retain the second section to the first section during adjustment of the second section.

30. An access port comprising:
a first section having a fixed cross section and a proximal end and a distal end defining a first path therethrough, the first path having a length and a width, and
an extension member operably coupled with the first section and adjustable relative to the first section to change the length of the first path, while the width of the first path remains fixed.

31. The access device of claim 30 wherein the first section has a generally tubular shape.

32. The access device of claim 30 wherein the first section has a plurality of stops for adjusting the length of the first path.

33. The access device of claim 32 wherein the extension member has a projection for engaging the plurality of stops of the first section.

34. The access device of claim 32 wherein the extension member has a flexible tab having a projection for engaging the plurality of stops of the first section.

35. The access device of claim 32 wherein the plurality of stops are teeth aligned longitudinally along the inner surface of the first section.

36. The access device of claim 30 wherein, the first section has a rail projecting and extending along the inner surface for coupling with the extension member.

37. The access device of claim 36 wherein the side surfaces of the extension member are contoured for engaging the rails of the first section.

38. The access device of claim 30 wherein the extension member has a chamfer at a distal end.

39. The access device of claim 30 wherein the distal end of the extension member has a contour to match an anatomical structure.

40. The access device of claim 30 wherein more than one extension member is operably coupled to the first section.

41. The method of accessing a surgical site using an adjustable access device comprising:
positioning through a skin incision to a surgical site proximate a vertebrae an adjustable access device having a first section with a fixed cross-section and an extension member operably coupled with the first section,
and adjusting the extension member within the incision relative to the first section to change the length of the first path, while the width of the first path remains fixed.

42. The method of claim 41 further comprising adjusting a number of extension members within the incision to adjust the length of the first path.

43. The method of claim 41 wherein the length of the first path is different along at least one extension member.

44. The method of claim 42 wherein adjusting a number of extension members includes adjusting at least one extension member to a different length than the other members.
